(19) United States
(12) Patent Application Publication
(10) Pub. No.: US 2005/0197682 A1
(43) Pub. Date: Sep. 8, 2005

(54) Method and apparatus for treating erectile sexual dysfunction using light energy

(76) Inventors: Sherry Fox, Parker, CO (US); Allen M. Sudduth, Arvada, CO (US)

Correspondence Address: JOHN R LEY, LCC, 5299 DTC BLVD, SUITE 610, GREENWOOD VILLAGE, CO, 80111, US

(21) Appl. No.: 10/791,653

(22) Filed: Mar. 2, 2004

(51) Int. Cl. A61N 5/06

(52) U.S. Cl. 607/88; 607/89

(57) ABSTRACT

Light energy, preferably at a wavelength of 884 nanometers, is applied to the genitalia area of male and female humans and animals to treat erectile sexual dysfunction. The light causes the release of nitric oxide into the erectile genitalia tissue, and the nitric oxide causes the smooth erectile tissue to relax and engorge, thereby facilitating erection. Separately structured applicators for males and females optimize the degree of light energy penetration and the amount of nitric oxide released according to the separate physiology of males and females. The light energy may be used to augment a pharmacologically-induced release of nitric oxide.

This invention relates the use of light energy to treat erectile sexual dysfunction in humans and animals. More particularly, the present invention relates to a new and improved method and apparatus for directing light energy, preferably infrared or near-infrared light energy, onto erectile genitalia tissues for the purpose of enhancing engorgement of those tissues and thereby diminish or eliminate this form of biological dysfunctional sexual response. The light is non-invasive and avoids or reduces the side effects and risks associated with pharmacologically-induced treatments of erectile sexual dysfunction, among other things.

### BACKGROUND OF THE INVENTION

Human sexual response is a complex mechanism involving both psychological and physiological response components. The psychological components relate to desire, interest, mood, and many other similar and related emotional factors. The physiological or biological response components of males and females differ because of physiology, but the common factor in both male and female is erection of genitalia tissue. Erection results from vasodilation of the vessels supplying blood to the genitalia tissues. Vasodilation increases the blood flow to the erectile genitalia tissues and results in engorgement of those tissues. In a male, dilation of the arteries leading to the corpus cavernosum of the penis causes engorgement with attendant enlargement and rigidity of the penis, while the enlargement constricts blood flow from the penis through venous channels. In a female, dilation of the blood vessels in the labia and in the tissue surrounding the vagina causes engorgement and erection of these tissues.

The engorgement of the erectile genitalia tissues results from the relaxation of smooth muscles of these tissues. The smooth muscles are normally in a contracted state to prevent engorgement from normal blood flow. In response to sexual stimulation, the smooth muscles relax, the blood flow to those tissue increases, and the genitalia tissues engorge and become erect. Smooth muscle relaxation is caused by a cascade of biological reactions and chemistries which occur as part of a normal sexual response. The release of nitric oxide is one of the cascade of biological reactions and chemistries leading to smooth muscle relaxation.

In a normal sexual response, nitric oxide is released from the nerve endings in the genitalia tissues. Nitric oxide is also bound to hemoglobin within red blood cells. It has also been theorized that the vascular smooth muscle in vivo may also accumulate some type of end product formed from endothelium-derived nitric oxide. The nitric oxide activates an enzyme called guanylate cyclasa, and that enzyme in turn helps elevate the level of cyclic guanosine monophosphate (cGMP). Cyclic guanosine monophosphate causes the smooth muscles to relax. Thus, the presence of nitric oxide assists in relaxing the smooth muscles of the erectile genitalia tissues, and thereby promotes their engorgement to achieve erection.

It is known that the application of light or photo energy to tissues and blood has the effect of influencing the localized release of nitric oxide, thereby stimulating vasodilation as a result of the effect of nitric oxide on the level of cyclic guanosine monophosphate. Light energy of specific wavelengths has the capability of freeing nitric oxide from the hemoglobin or otherwise releasing nitric oxide from the smooth muscle and vessels. The application of light energy to tissue to achieve therapeutic healing is also well-known. Light energy causes certain photoreactive enzymes to accelerate their functions, thereby enhancing cellular metabolism, circulatory improvement and nerve function, all of which contribute to healing.

A variety of different light emitting devices intended to be used for healing and therapeutic purposes, as well as a variety of different uses of such therapeutic devices, have been proposed. An example of one such device and several possible therapeutic applications of it are described in U.S. Pat. No. 6,471,716.

The most common treatment for erectile sexual dysfunction in men is pharmacological treatment. The typical pharmaceuticals currently available for male erectile dysfunction are presently marketed under the trademarks “Viagra,” “Levitra” and “Cialis.” These pharmaceuticals introduce certain chemicals into the body which are effective in increasing blood flow to the penis. Introduced chemicals also create certain undesirable side effects, such as fringe blurred and color-distorted vision, flushed face, stuffiness, headache, upset stomach and backache. Men with certain types of pre-existing diseases may not be able to accept erectile-dysfunction pharmaceuticals, because such pharmaceuticals may interfere with other medicines taken by to address some other pre-existing disease. In addition, effects of the pre-existing disease itself may be exacerbated by such erectile-dysfunction pharmaceuticals. More importantly, it may be impossible or extremely risky to attempt medical intervention for some types of diseases and afflictions, such as a heart attack, until the erectile-dysfunction treating pharmaceutical has been sufficiently metabolized within the individual to the point where it is no longer effective. The inability to intervene medically in such circumstances can be extremely serious, since many of the erectile-dysfunction pharmaceuticals are taken by older men who are more prone to such diseases and and afflictions.

### SUMMARY OF THE INVENTION

This invention applies light energy to increase nitric oxide and enhance vasodilation in the genitalia tissues, thereby diminishing or overcoming erectile sexual dysfunction. The invention is applicable to both males and females, as well as to humans and animals. Because the light application is noninvasive, the present invention may be used without inhibiting the ability to medically intervene in certain types of diseases and afflictions. Moreover, the light application of the present invention may be used in conjunction with erectile-dysfunction pharmaceuticals to achieve an overall enhanced effect that neither the pharmaceuticals nor the light could achieve by itself. Depending upon the individual circumstances, it may be possible that the application of the light energy is itself sufficient to overcome erectile sexual dysfunction or to at least diminish the effects of erectile sexual dysfunction to the point where adequate sexual activity can be achieved.

One aspect of the invention involves a method for treating erectile sexual dysfunction. The method is accomplished by applying light to the erectile genitalia tissue. A sufficient amount of light is applied to release nitric oxide into the erectile genitalia tissue, and the released nitric oxide is of sufficient quantities to cause the erectile tissue to relax and engorge. For males, the light is preferably applied to penetrate into the top of the penis and to preferably reach the corpus cavernosum of the penis. For females, the light is preferably applied to penetrate into the clitoris and labia and to encompass the general external area of the vagina.

Preferably, the applied light energy has a wavelength in the infrared or near-infrared range, most preferably at approximately 884 nanometers. The light energy is also preferably monochromatic, thereby concentrating the energy application at a single frequency or at a single narrow range of frequencies. Light of these wavelengths has the capability of penetrating deeply into tissue, and thereby reaching the smooth muscle tissue which controls blood flow into the erectile genitalia tissue. Pharmacologically-induced release of nitric oxide into erectile genitalia tissue may be augmented by applying the light to the genitalia tissue.

Another aspect of the invention involves an apparatus for treating erectile sexual dysfunction. The apparatus comprises a frame structure having an application surface of sufficient dimensions to encompass the erectile genitalia tissues, a plurality of light sources distributed in an array over the application surface, and a source of energy connected to energize the light sources. The energized light sources emit light energy of a wavelength sufficient to penetrate into the erectile genitalia tissues and release nitric oxide. The light preferably releases a sufficient amount of nitric oxide to cause the erectile genitalia tissue to relax and engorge. For males, the applicator surface defines an inner substantially-semicircular configuration having dimensions sufficient to surround the top of the penis. For females, the applicator surface has dimensions sufficient to encompass the clitoris and labia. Preferably, each light source comprises an infrared light emitting diode. The apparatus preferably comprises a control module which includes a source of energy that is supplied to the light emitting diodes through a cable.

A more complete appreciation of the scope of the present invention and the manner in which it achieves the above-noted and other improvements can be obtained by reference to the following detailed description of presently preferred embodiments taken in connection with the accompanying drawings, which are briefly summarized below, and by reference to the appended claims.

### BRIEF DESCRIPTION OF THE DRAWINGS

FIG. 1 is a flow chart illustrating the application of light energy for the treatment of erectile sexual dysfunction in accordance with the present invention.

FIG. 2 is a perspective view of an applicator and a control module used by males to apply light energy to male genitalia in accordance with the treatment shown in FIG. 1.

FIG. 3 is a perspective view of the applicator shown in FIG. 2, the applicator shown inverted from its view shown in FIG. 2.

FIG. 4 is a perspective view of an applicator used by females to apply light energy to female genitalia in accordance with the treatment shown in FIG. 1.

### DETAILED DESCRIPTION

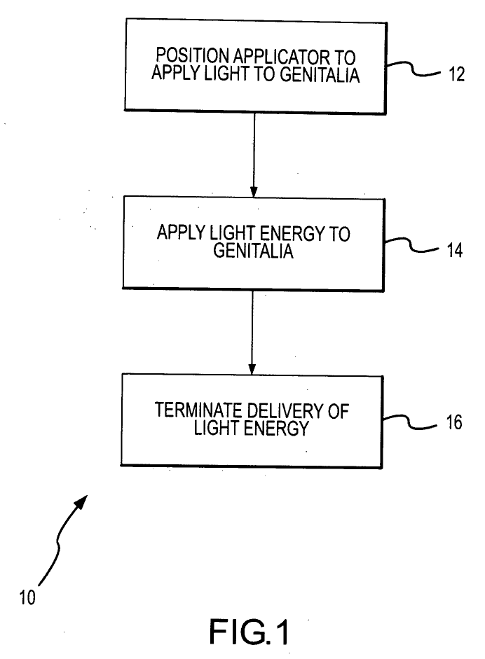

A method 10 of treating erectile dysfunction in males and females is generally illustrated in FIG. 1. The method 10 commences by positioning an applicator on, over or surrounding the male or female erectile genitalia tissues, as shown at 12. Once the applicator is positioned, light energy is applied from the applicator to the erectile genitalia tissue, as shown at 14. In general, the light energy is delivered as a result of energizing a light source which causes light energy to emanate from the applicator on the erectile genitalia tissues. The light energy is applied at 14, until the further delivery of light energy is terminated as shown at 16.

The light energy is applied at 14 for the amount of time required to achieve the desired level of assistance with the erection. The light energy is applied to cause the release of nitric oxide, which assists in causing the smooth tissues to relax, thereby permitting the erectile genitalia tissues to engorge. The engorgement resulting from the light-induced presence of nitric oxide assists in developing and maintaining an erection. The light energy applied to the genitalia may be sufficient by itself to assist in developing and maintaining the erection, or the light energy may augment the effect of erection-promoting pharmaceuticals ingested by the male or female.

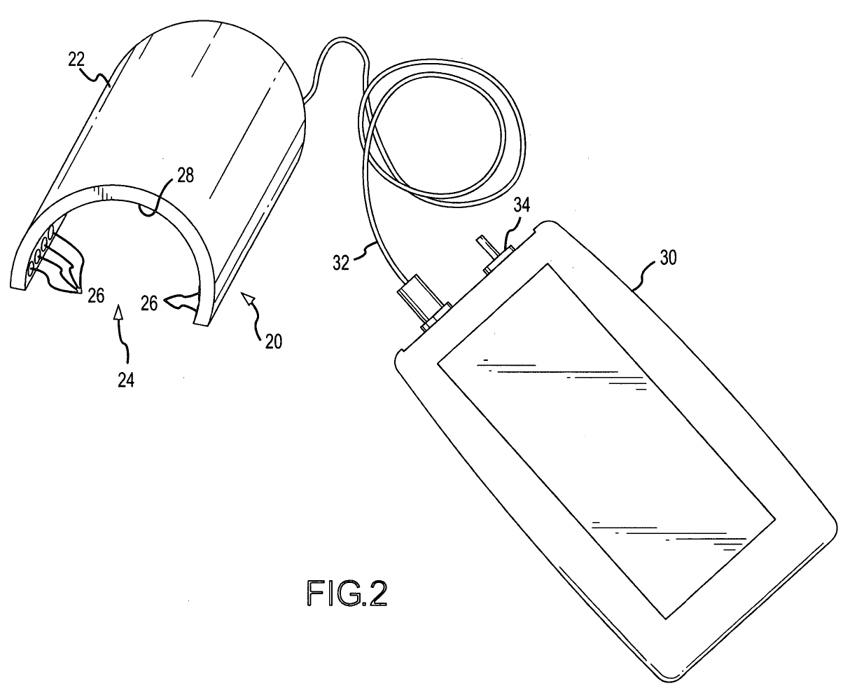

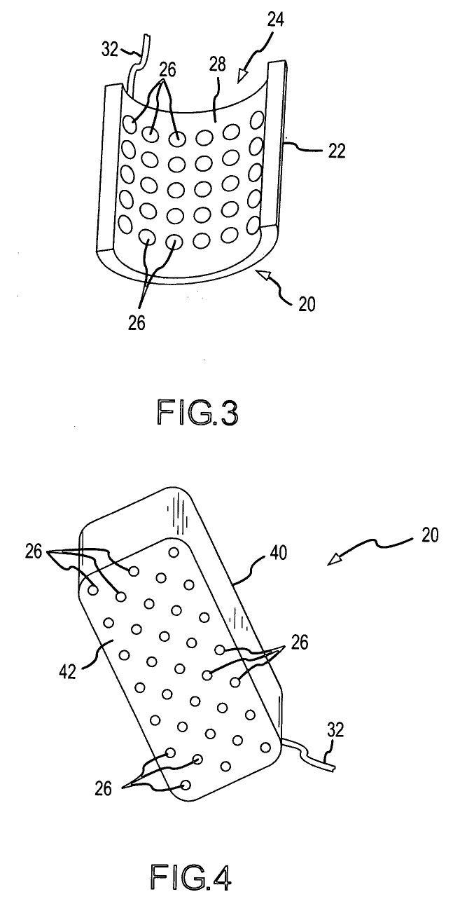

The preferable form of an applicator 20 used by males is shown in FIGS. 2 and 3. The applicator 20 includes a frame structure 22 which defines a generally inward-opening semicircular channel 24 which extends along the length of the frame structure 22. The semicircular channel 24 generally takes the shape of a cylinder which has been severed diametrically and parallel to the axis of the cylinder. The material from which the frame structure 22 is formed may either be rigid or flexible.

A plurality of light emitting sources 26 are located on an inner semicircular application surface 28 which is defined by the channel 24. Preferably, each of the light sources 26 comprises a light emitting diode (LED), preferably which emits infrared or near infrared light energy at a wavelength of approximately 884 nanometers. Wavelengths of approximately 884 nanometers have been found to achieve the maximum depth of penetration into typical vascular animal tissue. U.S. Pat. No. 6,471,716 describes a thermal feedback technique of shifting the normal 880 nanometer wavelength of certain types of light emitting diodes to a higher 884 nanometer wavelength, and such a technique may be utilized in this invention.

The light sources 26 are preferably organized in arrays. Preferably, the light sources 26 are positioned in separate linear arrays, with each linear array extending parallel to the axis of curvature of the semicircular surface 28. The number of linear arrays of light sources 26 incorporated in the frame structure 22 is selected to deliver the desired or maximum amount of light into the channel 24. Each light source 26 of adjoining linear arrays is preferably longitudinally offset with respect to one another. In this manner, as many light sources 26 as possible may be incorporated along the semicircular surface 28.

Energy for the light sources 26 is supplied by a control module 30, as shown in FIG. 2. The control module 30 includes or is used with an energy source, such as a battery or a converter for converting conventional AC mains electrical energy into energy suitable for powering the light sources and the module 30. A cable 32 conducts the energy from the module 30 to the light sources 26. The control module 30 also includes a power switch 34. Activation of the power switch 34 causes the control module 30 to become operative and to deliver energy from the energy source over the cable 32 to energize the light sources 26.

The cable 32 may contain a plurality of individual electrical conductors by which to conduct current to the light sources 26 or to the linear arrays of the light sources 26. In this manner, one or more of the light sources 26 or the linear arrays may be energized simultaneously, or the linear arrays may be energized in an alternating or rotational manner. U.S. Pat. No. 6,471,716 describes a technique for individually energizing arrays of light emitting diodes in a repetitive rotational sequence, and such a technique may be utilized by the control module 30. However, all of the light sources 26 of the applicator 20 may be energized simultaneously and continuously, as well, or individual ones of the light sources 26 may be individually energized.

As an alternative to light emitting diodes or other light generating sources, the light sources 26 may comprise an optical lens which directs the light energy into the channel 24. In such a case, the light source 26 may not itself generate the light. For example, the cable 32 may include a plurality of light conducting fiber optic cables, with each of the fiber-optic cables transmitting light from a light source within the control module 30 to the optical lens. Alternatively, light generating sources could be included within the frame structure 22, and the light from those sources divided and transmitted to the light sources 26 by optical transmitting guides located within the frame structure 22. In yet another alternative, the frame structure 22 could include optical elements which divide the light from a single optical fiber within the cable 32, and that divided light is then distributed to the optical lenses. In general, however, the preferred form is to incorporate a light generating emitter in each source 26, and to electrically energize that light generating emitter to cause it to emit light, in much the same way that a light emitting diode operates.

The frame structure 22 defines the downward facing semicircular channel 24 for the purpose of allowing the channel 24 to be placed over the top or penile dorsum of the male penis or male genitalia. The length of the channel 24 provided by the frame structure 22 is sufficient to cover most of the top area from the base of the penis to the glans or head of the penis, under most or all states of erection. The frame structure 22 may be sufficiently flexible or resilient to permit the channel 24 to follow the curvature of the penis along its length and to increase or decrease the transverse width of the channel 24. The light sources 26 are preferably incorporated as a smooth continuation of the inner semicircular surface 28 of the frame structure 22, to permit the light sources 26 to deliver the light energy directly into the top of the penis. In the case of light emitting diodes which emit light at 884 nanometers, positioning the light emitting diodes and the contact surface 28 closely adjacent to, or in contact with, the top tissue of the penis causes maximum penetration of the light energy into the erectile tissues along the top of the penis.

The tissues along the top of the penis are primarily responsible for erection. Two large cylinder-like columns of smooth erectile tissue extend along the top of the penis from the base to the glans or head of the penis. These smooth erectile tissues are separated by a septum of fibers. Together, these two columns of smooth erectile tissues constitute the corpus cavernosum. The blood flows into and fills up the corpus cavernosum to create the erection. The light energy applied directly to the top of the penis penetrates into the these two columns of smooth erectile tissues to release nitric oxide at the location where it has the greatest effect on relaxing those erectile tissues and allowing them to engorge with blood. As the corpus cavernosum enlarges due to engorgement, the veins on the lower or ventral side of the penis are constricted by the engorgement, and the constricted veins resist venous drainage of blood from the penis and assist in maintaining the blood in the corpus cavernosum.

The preferable form of the applicator 20 used by females is shown in FIG. 4. In this case, a frame structure 40 of the applicator 20 is generally rectangularly shaped. The light sources 26 are positioned on a substantially rectangular application surface 42. The light sources 26 are preferably arranged in linear arrays on the rectangular surface 42, with the individual light sources of each line transversely offset from one another to enable the maximum number of light sources on the application surface 42. The frame structure 40 is connected to the control module 30 by the cable 32, and energy is applied from the control module 30 over the cable 32 by the power switch 34 in the same or similar manner as has been previously described in conjunction with FIGS. 2 and 3. In general, all of the energizing and positioning considerations of the light sources 26, which have previously been discussed in conjunction with the male form of the applicator 20 shown in FIGS. 2 and 3, also apply with respect to the female form of the applicator shown in FIG. 4.

The width of the application surface 42, and the width of the light sources 26 on the surface 42, are established to encompass the clitoris and labia at the vagina of the female genitalia. The application surface 42 is placed directly over on top of the clitoris and labia, and preferably in physical contact with the clitoris and labia, so that the light from the sources 26 will attain the maximum depth of penetration. The application surface 42 may be curved in a slightly concave manner, rather than being planar, to facilitate the closest positioning and contact of the light sources 26 over and with the clitoris and labia, thereby transferring the light energy more deeply into these female genitalia tissues.

The light energy penetrating into the female genitalia tissues causes nitric oxide to be released, thereby relaxing the smooth erection tissues and allowing them to engorge with blood. The dilation of the blood vessels and the engorgement of the labia and the tissue surrounding the vagina produce an area at the distal third of the vagina where the blood collects. Subsequently, ballooning of the proximal portion of the vagina and elevation of the uterus occurs to complete the erection. Applying the light energy to the clitoris and the area surrounding the labia assures that released nitric oxide will have the effect of relaxing the smooth erectile tissues to enhance their susceptibility to engorgement.

The shapes and configurations of the application surfaces 28 and 44 may be varied to more closely conform with the male and female genitalia anatomy, thereby increasing the transfer efficiency of the light energy into the erectile tissues. Although not necessarily as efficient in the transfer of light energy, a single shape and configuration for an application surface may prove sufficiently satisfactory for use by both males and females. In some circumstances, the control module 30 may be incorporated within the frame structures 22 and 40, along with a battery power supply, thereby creating a single, self contained and internally powered device capable of delivering the light energy to the male and female erectile genitalia tissues.

Light energy at 884 nanometers has been found to offer the best penetration into moderately vascular tissues, such as the male and female erectile genitalia tissues. An 884 nanometer wavelength is infrared, and accordingly, it is preferred that the infrared light emitting diodes emit light energy at 884 nanometers. However, other near-infrared frequencies which result in the release of nitric oxide to promote relaxation of the erectile tissues and allow them to engorge with blood, are also suitable for use with the present invention.

In the manner described above, applying light energy to the male and female genitalia has the effect of enhancing or promoting erection. The light energy is noninvasive, and a beneficial effect on overcoming erectile sexual dysfunction is achieved without pharmacological intervention. However, because of the noninvasive nature of the light application, the light may be used to augment or to enhance the erection effects achieved by pharmaceuticals. The use of light to facilitate erection does not carry the side effects of pharmaceuticals, and does not place the users at risk of denial of certain types of medical treatment or intervention after having taken erection-enhancing pharmaceuticals. In addition the use of the present invention has the potential to reduce the consumed amount of such erection-enhancing pharmaceuticals necessary to achieve a satisfactory erection. Other advantages of the use of light energy to enhance and promote erection will become known upon appreciation of the present invention.

A presently preferred embodiment of the present invention and many of its improvements have been described with a degree of particularity. This description is a preferred example of implementing the invention, and is not necessarily intended to limit the scope of the invention. The scope of the invention is defined by the following claims.

1. A method of treating erectile sexual dysfunction by applying light to erectile genitalia tissue.

2. A method as defined in claim 1, further comprising applying infrared light to the genitalia tissue.

3. A method as defined in claim 1, further comprising applying near-infrared light to the genitalia tissue.

4. A method as defined in claim 1, further comprising applying substantially monochromatic light having a wavelength of approximately 884 nanometers to the genitalia tissue.

5. A method as defined in claim 1, further comprising applying a sufficient amount of light to release nitric oxide into erectile genitalia tissue.

6. A method as defined in claim 1, further comprising applying a sufficient amount of light to release nitric oxide into erectile genitalia tissue in sufficient quantities to cause the erectile tissue to relax and engorge.

7. A method as defined in claim 1, further comprising augmenting a pharmacologically-induced release of nitric oxide into erectile genitalia tissue by applying the light to the genitalia tissue.

8. A method as defined in claim 7, further comprising applying a sufficient amount of light to release nitric oxide into erectile genitalia tissue that would otherwise not be released pharmacologically.

9. A method as defined in claim 1, applied to males.

10. A method as defined in claim 9, further comprising applying the light to penetrate into the corpus cavernosum of the penis.

11. A method as defined in claim 9, further comprising applying the light to penetrate into the top of the penis.

12. A method as defined in claim 11, further comprising applying the light from an applicator having a plurality of light sources distributed in an array over an inner substantially-semicircular application surface having dimensions sufficient to surround the top of the penis, and positioning the application surface over the top of the penis.

13. A method as defined in claim 12, further comprising positioning the application surface against the top of the penis.

14. A method as defined in claim 13, applied to humans.

15. A method as defined in claim 1, applied to females.

16. A method as defined in claim 15, further comprising applying the light to penetrate into external vagina tissues.

17. A method as defined in claim 16, further comprising applying the light to penetrate into the clitoris and labia.

18. A method as defined in claim 17, further comprising applying the light from an applicator having a plurality of light sources arranged in an array distributed over an application surface having dimensions sufficient to encompass the clitoris and labia, and positioning the application surface over the clitoris and labia.

19. A method as defined in claim 18, further comprising positioning the application surface against the labia.

20. A method as defined in claim 18, further comprising positioning the application surface against the clitoris.

21. A method as defined in claim 18, further comprising positioning the application surface against the clitoris and the labia.

22. A method as defined in claim 18, applied to humans.

23. Apparatus for treating erectile sexual dysfunction of an animal, comprising:
a frame structure having an application surface of sufficient dimensions to encompass erectile genitalia tissues of the animal;
a plurality of light sources distributed in an array over the application surface; and
a source of energy connected to energize the light sources; and wherein:
the energized light sources emit light energy of a wavelength sufficient to penetrate into the erectile genitalia tissues and release nitric oxide.

24. Apparatus as defined in claim 23, wherein:
the energized light sources emit light energy to release a sufficient amount of nitric oxide into the erectile genitalia tissue to cause the erectile genitalia tissue to relax and engorge.

25. Apparatus as defined in claim 23, wherein:
the energized light sources emit substantially monochromatic light at a wavelength of approximately 884 nanometers.

26. Apparatus as defined in claim 23, wherein:
the light sources comprise at least one light emitting diode.

27. Apparatus as defined in claim 23, wherein:
each light source comprises a light emitting diode which emits infrared light when energized, each light emitting diode attached to the frame structure; and further comprising:
a control module which comprises the source of energy, the source of energy comprising a source of electrical energy; and
a cable connecting the control module to the frame structure and conducting electrical energy from the electrical energy source to the light emitting diodes.

28. Apparatus as defined in claim 23 for use with males, wherein:
the applicator surface of the frame structure defines an inner substantially-semicircular configuration having dimensions sufficient to surround the top of the penis.

29. Apparatus as defined in claim 28, wherein:
the dimensions of the substantially-semicircular configuration allow the application surface to contact substantially the entire top of the penis between the base and the glans.

30. Apparatus as defined in claim 23 for use with females, wherein:
the application surface of the frame structure has dimensions sufficient to encompass the clitoris and labia.

31. Apparatus as defined in claim 30, wherein:
the dimensions of the application surface allow the application surface to contact simultaneously substantially the entire female genitalia area which includes the clitoris and labia.
